(19) United States
(12) Patent Application Publication
(10) Pub. No.: US 2004/0185018 A1
(43) Pub. Date: Sep. 23, 2004

(54) Nail enamelcompositions and methods

(76) Inventors: Anjali Abhimanyu Patil, Westfield, NJ (US); Robert Walter Sandewicz, Monroe Township, NJ (US); Warline L. Dullas, East Brunswick, NJ (US); Daniel Anthony Betancourt, Edison, NJ (US); Frank Charles Pagano, Monroe Township, NJ (US); Joseph Frank Calello, Bridgewater, NJ (US)

Correspondence Address: Julie Blackburn, Revlon Consumer Products Corporation, 237 Park Avenue, New York, NY, 10017, US

(21) Appl. No.: 10/770,068

(22) Filed: Feb. 2, 2004

Related U.S. Application Data
(60) Provisional application No. 60/444,547, filed on Feb. 3, 2003.

(51) Int. Cl. A61K 7/04

(52) U.S. Cl. 424/61

(57) ABSTRACT

Nail enamel compositions containing a monoester containing at least one alkyl ether group; or an ester of benzoic acid, or a diester formed by the reaction of a dicarboxylic acid where the two carboxylic acid groups are separated by a straight or branched chain alkyl group or an aryl group, and an aliphatic or aromatic alcohol and said diester further comprising at least one alkyl ether group; or a triester formed by the reaction of aromatic tricarboxylic acid and an aliphatic or aromatic alcohol, or by the reaction of a polyol having primary hydroxyl groups with an aliphatic carboxylic acid; and a method for reducing the hardness of one or more film forming polymers in a nail enamel composition, and/or improving nail enamel wear comprising adding such compounds to the nail enamel composition.

This application claims priority from provisional patent application serial No. 60/444,457, filed Feb. 3, 2003.

### TECHNICAL FIELD

The invention is in the field of nail enamel compositions and methods for improving wear of nail enamel compositions.

### BACKGROUND OF THE INVENTION

A large percentage of modem women regularly wear nail enamel. Prior to the invention of nail enamel, women typically cared for their nails by filing them into the desired shape—styles have varied from pointed to square tips throughout the years—and sometimes buffed them with colored creams to provide subtle color. In the 1920's nail enamel as we know it today became available in technology borrowed from the paint industry. Only lightly tinted shades were available, and “moon manicures”, where polish was applied only to the center of the nail and not the nail moon, were in vogue. Eventually nail enamel formulated with pigments rather than dyes was developed, and full ranges of colors became available.

Today basic nail enamel is generally comprised of one or more film forming polymers in a solvent medium, and, if desired, pigments or colorants. The liquid composition is painted onto the nails and the solvent evaporates leaving the dried nail enamel film on the nail. It is the quality of the dried film on the nail that dictates characteristics like wear, adhesion, gloss—all of which are physical characteristics that determine success or failure of the product with the consumer. It is important that the film be semi-permanent; meaning that it stays on the nails for a set period of time while the wearer engages in normal activities, but yet can be removed by remover compositions readily available to consumers. Desirable nail enamel films must also exhibit a certain degree of hardness otherwise-they will not form a suitable semi-permanent covering thereon. On the other hand, the film must not be too hard, or brittle, otherwise it will readily chip off the nail, sometimes in large sheets. One of the most important aspects of formulating commercially acceptable nail enamels is to ensure that the film formed on the nail is just the right hardness to provide optimum wear and comfort. In general, the film forming polymers traditionally used in nail enamel—such as nitrocellulose and various acrylate and methacrylate esters—provide films that are too hard when used alone. Accordingly it is necessary to incorporate other ingredients into the nail enamel to modulate the hardness of the film formed by the film forming polymer.

It is an object of the invention to provide a method for reducing the hardness of nail enamel film by incorporating into a nail enamel composition certain compounds that have ester and/or ether moieties.

It is a further object of the invention to provide a method for improving the wear of a nail enamel composition comprising incorporating into the nail enamel composition certain compounds that have ester and/or ether moieties.

It is a further object of the invention to provide a nail enamel composition comprised of at least one solvent, at least one film forming polymer, and at least one compound containing ester and/or ether moieties.

### BACKGROUND OF THE INVENTION

The invention is directed to a nail enamel composition comprising at least one solvent, at least one film forming polymer, and at least one compound selected from the group consisting of (“Compound”):

(a) a monoester containing at least one alkyl ether group,

(b) a monoester of benzoic acid and a long chain alcohol or ester alkanol, or fatty oil,

(c) a diester formed by the reaction of a dicarboxylic acid where the two carboxylic acid groups are separated by a straight or branched chain alkyl group or an aryl group, and an aliphatic or aromatic alcohol; said diester further comprising at least one alkyl ether group.

(d) a triester formed by (i) the reaction of aromatic tricarboxylic acid and an aliphatic or aromatic alcohol, or (ii) the reaction of a polyol having primary hydroxyl groups with an aliphatic carboxylic acid; and

(e) and mixtures thereof.

The invention comprises a method for reducing the hardness of one or more film forming polymers in a nail enamel composition, and/or improving nail enamel wear comprising adding to the nail enamel composition a Compound selected from the group consisting of:

(a) a monoester containing at least one alkyl ether group,

(b) a monoester of benzoic acid and a long chain alcohol or ester alkanol or fatty oil,

(c) a diester formed by the reaction of a dicarboxylic acid where the two carboxylic acid groups are separated by a straight or branched chain alkyl group or an aryl group, and an aliphatic or aromatic alcohol; said diester further comprising at least one alkyl ether group.

(d) a triester formed by (i) the reaction of aromatic tricarboxylic acid and an aliphatic or aromatic alcohol, or (ii) the reaction of a polyol having primary hydroxyl groups with an aliphatic carboxylic acid; and

(e) and mixtures thereof.

### DETAILED DESCRIPTION

I. The Compositions

The nail enamel compositions of the invention comprise at least one solvent, at least one film forming polymer, and at least one compound. With respect to the ingredients set forth herein, all percentages are by weight unless otherwise indicated.

A. Solvent

A wide variety of solvents may be suitable, including either aqueous or non-aqueous solvents or a combination of both. Suggested ranges are from about 0.1-99%, preferably about 0.5-85%, preferably about 5-80%. Non-aqueous solvents include short and/or medium chain (C₁₋₁₂) aliphatic or aromatic ketones such as acetone, diacetone alcohol, dihydroxyacetone, ethyl butyl valerolactone, methyl ethyl ketone, and the like; short and/or medium chain aliphatic or aromatic alcohols such as methanol, propanol, benzyl alcohol, butoxyethanol, butoxypropanol, isopropanol, butyl alcohol, 3-methyl-3-methoxy-butanol, t-butyl alcohol, butylene glycol, diethylene glycol, abietyl alcohol, cyclic ethers such as propylene carbonate, hexyl alcohol, isopropanol, and the like; glycol ethers; esters such as butyl acetate, ethanol, ethyl acetate 1-methoxy-2-propanol acetate; benzoates and the like.

B. The Film Forming Polymer

A wide variety of film forming polymers may be used in the compositions of the invention. The term “film forming polymer” means a polymer that is soluble or dispersible in the solvent of the nail enamel composition and is capable of remaining soluble or dispersible, without settling out in the composition during the shelf life of the product. At the same time, when the composition is applied to the nails and the solvent dries, the polymer should be capable of forming a semi-permanent adherent film on the nails, preferably, having a thickness ranging from about 0.1 to 10 mils (thousandths of an inch). The film forming polymer is further characterized in that once it is applied to nails in the form of a nail enamel composition, the film cannot be removed with water alone, but rather is removable only with organic solvent-based removers containing ingredients such as acetone, butyl acetate, ethyl acetate, methyl ethyl ketone, and the like.

The film forming polymers may be natural or synthetic, or a combination of both, and may be in the form of solids, semi-solids, or liquids. The film forming polymer may be neutral or ionic in character, e.g. anionic, cationic, nonionic, or amphoteric. Suggested ranges of film forming polymer are about 0.1-85%, preferably about 2-80%, more preferably about 5-70% by weight of the total composition.

1. Synthetic Polymers

(a). Polymers Made From Ethylenically Unsaturated Monomers

Suitable synthetic polymers include homopolymers, copolymers, and block and graft copolymers made by polymerizing ethylenically unsaturated monomers, including acrylic or methacrylic acid or their simple esters or amides, styrene, vinyl, and so on. Examples of synthetic film forming polymers include those set forth in the CTFA Cosmetic Ingredient Dictionary and Handbook, Eighth Edition, 2000, pages 1744 through 1747, which are hereby incorporated by reference, including those that are summarized herein. Certain other types of synthetic polymers that may be used in the nail enamel compositions include those comprised of one or more monomers selected from the following general formula:

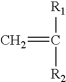

wherein R₁ is H, a C₁₋₃₀ straight or branched chain alkyl, aryl, arylalkyl; R₂ is a pyrrolidone, or a substituted or unsubstituted aromatic, alicyclic, or bicyclic ring where the substitutents are C₁₋₃₀ straight or branched chain alkyl, or COOM wherein M is H, a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more halogens.

Even more preferred as synthetic polymers, which comprise polar monomers such as acrylic acid or methacrylic acid, in combination with C₁₋₆ esters thereof. Most preferred is a synthetic polymer that comprises monomers of butyl methacrylate and acrylic acid.

(b). Copolymers of Silicone and Organic Monomers

Also suitable for use as the film forming polymer in the compositions and method of the invention are copolymers of silicone and various organic, ethylenically unsaturated monomers, and optionally other monomers. Examples of such polymers are disclosed in U.S. Pat. No. 6,033,650, which is hereby incorporated by reference. Preferred examples of these polymers include graft or block copolymers comprised of silicon moieties and C₁₋₁₂ alkyl acrylate or methacrylate monomers which may be substituted with one or more groups such as halogen or hydroxy, also referred to as silicone/acrylate copolymers. Suitable silicone acrylate copolymers may be purchased from 3M Company under the tradenames VS-70 and SA-70, or from Shin Etsu Silicones.

(c). Urethane Homo- and Copolymers

Also suitable for use in the compositions and method of the invention are homo and copolymers of urethane. Homopolymers of urethane are often sold in an aqueous dispersion from vendors such as Allied Colloids, B.F. Goodrich, and the like. Typically, urethane polymers are obtained by polymerizing carbamate or urethane groups (—NHCOO—) either alone or in combination with other types of monomers. Suitable urethane copolymers may be comprised of urethane monomers copolymerized with ethylenically unsaturated monomers as mentioned herein (such as acrylic acid, methacrylic acid or their simple esters, styrene, vinyl). Similarly, the urethane monomers may be copolymerized with one or more different organic compounds such as mono-, di-, or polyhydric alcohols, C₁₋₁₀ alkylene glycols, fatty alcohols, and the like.

(d). Amides and Amines

Also suitable are various synthetic polymers containing amide or amine substituent groups. Examples of such polymers include nylon, ammonium polyacrylate, acrylamides copolymer, acrylates/acrylamide copolymers, acrylates ammonium acrylate copolymer, acrylates C10-20 alkyl acrylate cross polymer, acrylates/carbamate crosspolymer, acrylates ceteth-20 itaconate copolymer, acrylates/dimethylaminoethyl methacrylate copolymer, ammonium acrylates copolymer, ammonium polyacrylate, ammonium styrene/acrylates copolymer, ammonium vinyl acetate/acrylates copolymer, aminomethylpropanol/acrylates/dimethylaminoethylmethacrylate copolymer, and so on.

2. Natural Polymers

A variety of natural polymers, or derivatives thereof are suitable, including cellulosics, chitins, chitosans, shellac, rosins, resins, animal or vegetable proteins and polypeptides, and so on. The natural polymers may be present in ranges from about 0.1-95%, preferably about 1-85%, more preferably about 3-45% by weight of the total composition.

(a). Cellulosics

Examples of suitable cellulosic polymers include nitrocellulose, mono- or diesters of cellulose formed by the reaction of cellulose with various organic acids, for example straight or branched chain carboxylic acids having from one to twenty, preferably one to ten carbon atoms, which may be substituted with one or more hydroxyl groups, Examples of such cellulosics include cellulose acetate, cellulose acetate isobutyrate, cellulose acetate propionate, cellulose acetate propionate carboxylate. Also suitable are cellulose polymers prepared by reacting with groups such as hydroxyl, alkoxyalkyl, hydroxylalkyl where the alkoxyalkyl and alkyl groups have from about one to ten carbon atoms. Examples of such polymers are carboxylmethyl hydroxyethylcellulose, carboxymethylcellulose, ethyl cellulose, hydroxyethylcellulose, methyl ethylcellulose, hydroxypropylcellulose, hydroxylbutyl cellulose, hydroxybutyl methylcellulose, and so on.

It may be desirable to have more than one film forming polymer in the composition. They polymers may be a combination of one or more synthetic polymers, or one or more natural polymers, or mixtures of both.

C. The Compound

The Compound incorporated into the nail enamel compositions and used in the method is selected from the group consisting of:

(a) a monoester containing at least one alkyl ether group,

(b) a monoester of benzoic acid and a long chain alcohol or alkoxy alkanol, or fatty oil,

(c) a diester formed by the reaction of a dicarboxylic acid where the two carboxylic acid groups are separated by a straight or branched chain alkyl group or an aryl group, and an aliphatic or aromatic alcohol, said diester further comprising at least one alkyl ether group.

(e) a triester formed by (i) the reaction of aromatic tricarboxylic acid and an aliphatic or aromatic alcohol, or (ii) the reaction of a polyol having primary hydroxyl groups with an aliphatic carboxylic acid; and

(f) mixtures thereof.

Suggested ranges of Compound are from about 0.01-60%, preferably about 0.05-55%, more preferably about 0.1-50% by weight of the total composition.

1. The Monoester

One of the Compounds may be a monoester containing at least one alkyl ether group where the alkyl ether group may be found on the aliphatic or aromatic carboxylic acid or the aliphatic or aromatic alcohol that are reacted to form the monoester. More preferred are monesters containing at least one alkyl ether group, and which is formed by the reaction of a C₁₋₁₀₀, preferably a C₁₋₄₈ aliphatic or aromatic carboxylic acid, and a C₁₋₁₀₀ aliphatic or aromatic alcohol containing the at least one alkyl ether group. The alkyl ether group may be a single alkyl ether group or a repeating alkyl ether group. Preferably, the alkyl ether groups include ethoxy, propoxy, isopropoxy, butoxy, isobutoxy, or combinations thereof.

More preferred is where the monoester is formed by the reaction of a C₁₋₂₀ aliphatic or aromatic carboxylic acid, and a C₁₋₁₀₀ aliphatic alcohol having at least one ethoxy, propoxy, isopropoxy, butoxy, or isobutoxy group or combinations thereof. One preferred monoester comprises one to four, preferably one, repeating propoxy group, and one to twenty, preferably three, repeating ethoxy groups and is the reaction of acetic acid and an ethoxylated, propoxylated cetyl alcohol.

Most preferred is where the monoester is propylene glycol isoceteth-3 acetate having the formula:

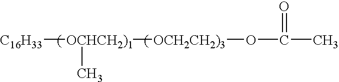

Another preferred monoester is the ester of propylene glycol myristyl ether and acetic acid, which is propylene glycol myristyl ether acetate. This ester is commercially available from suppliers such as Bernel Chemical Company.

Another preferred monoester is formed by the reaction of an aromatic acid or alcohol (such as benzoic acid or benzyl alcohol) and a C₁₋₁₀₀ aliphatic or aromatic alcohol or carboxylic acid where the alkyl ether group may be on the aromatic acid or alcohol, or the aliphatic or aromatic carboxylic acid. More preferred is a monoester of benzoic acid and a C₁₋₅₀ aliphatic alcohol having one or more repeating alkyl ether groups (for example, from 1 to 100 repeating alkyl ether groups) selected from ethoxy, propoxy, isopropoxy, butoxy, isobutoxy, and mixtures thereof. Particularly preferred is where the compound is the reaction product of benzoic acid and a C₆₋₅₀ fatty alcohol such as stearyl alcohol, having from two to twenty, preferably about 15, repeating propoxy groups and the compound is PPG-15 stearyl ether benzoate. One example of such a Compound has the formula:

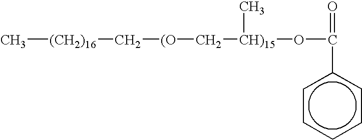

Another preferred monoester suitable for use in the compositions of the invention is the reaction product of an aromatic alcohol (such as benzyl alcohol) and a

C₁₋₁₀₀ aliphatic carboxylic acid where the alkyl ether group may be found on the acid or alcohol. More particularly, the monoester is the reaction product of benzyl alcohol and a

C₆₋₅₀ fatty aliphatic carboxylic acid substituted with one or more alkyl ether groups. Preferably the alkyl ether groups are repeating alkyl ether groups selected from ethoxy, propoxy, isopropoxy, butoxy, isobutoxy, and mixtures thereof. Most preferred is a monoester formed by the reaction of propoxylated benzyl alcohol with myristic acid, which is PPG-3 benzyl myristate.

2. The Monoester of Benzoic Acid

Another Compound that may be used in the compositions of the invention is a monoester of benzoic acid and a long chain alcohol or ester alkanol, or fatty oil such as castor oil. The term “long chain” when used herein means an alcohol having greater than 4 carbon atoms, preferably from about 6 to 50 carbon atoms, or in the case of the ester alkanol, that the alkanol portion has greater than 4 carbon atoms, and preferably from 6 to 50 carbon atoms.

One type of such Compound comprises a monoester of benzoic acid and a long chain alcohol. Preferably the long chain alcohol has from about 6 to 50 carbon atoms. Examples of such alcohols include stearyl alcohol, ethylhexyl alcohol, C₁₂₋₁₅ alcohols, myristyl alcohol, cetyl alcohol, isostearyl alcohol, oleyl alcohol, and the like. Particularly preferred esters of benzoic acid and long chain alcohols such as ethylhexylbenzoate, isostearyl benzoate, C₁₂₋₁₅ alkyl benzoate, cetyl benzoate, stearyl benzoate, and so on.

Another type of benzoic acid ester suitable for use in the compositions of the invention is a benzoic acid ester which is formed by the reaction of benzoic acid and an ester alkanol where the ester alkanol may be saturated, or a mixture of saturated and unsaturated carbon atoms. Particularly suitable are benzoic acid esters formed by the reaction of benzoic acid and an ester alkanol formed by the reaction of an acid and an alcohol where the alcohol or the acid may have one or more hydroxyl groups, such as a fatty (C₆₋₃₀) acid and a fatty (C₆₋₃₀) alcohol. Examples of such benzoic acid esters include cetyl ricinoleate benzoate, cetylstearyl benzoate, cetyl isostearyl benzoate. In the case of cetyl ricinoleate benzoate, the ester cetyl ricinoleate, which has a hydroxyl group, reacts with benzoic acid to form cetyl ricinoleate benzoate.

Yet another type of benzoic acid ester suitable for use in the compositions of the invention is the ester of benzoic acid and a fatty oil such as castor oil. Particularly preferred is castor oil benzoate, which is the ester formed by the reaction of benzoic acid with castor oil.

3. The Diester

The Compound may also be a diester formed by the reaction of a dicarboxylic acid where the two carboxylic acid groups are separated by a straight or branched chain alkyl group or an aryl group, and an aliphatic or aromatic alcohol, and said diester further comprising at least one alkyl ether group. The alkyl ether group may be found on the dicarboxylic acid or the aliphatic or aromatic alcohol. Preferably, the diester is formed by the reaction of a dicarboxylic acid wherein the two carboxylic acid groups are separated by a straight or branched chain alkyl group containing from one to forty, preferably one to twenty, carbon atoms and the alcohol is an aliphatic alcohol containing at least one alkyl ether group. Preferred is where the aliphatic alcohol contains from one to forty carbon atoms and the alkyl ether group is a repeating alkyl ether group and comprises ethoxy, propoxy, isopropoxy, butoxy, isobutoxy, or combinations thereof. Particularly preferred is where the diester is formed by the reaction of a dicarboxylic acid where the two carboxylic acid groups are separated by a C₁₋₁₀ straight chain alkyl and the alkyl ether group is propoxy, in particular wherein both carboxylic acid groups of the dicarboxylic acid are reacted with an aliphatic alcohol containing a repeating alkyl ether group which is ethoxy, propoxy, isopropoxy, or combinations thereof.

One preferred diester is formed by the reaction of adipic acid and myristyl alcohol containing from one to four repeating propoxy groups and wherein the compound is di-PPG-3 myristyl ether adipate having the formula:

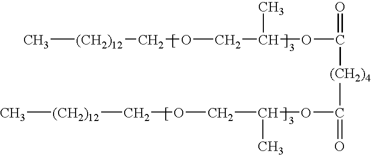

Another preferred diester is formed by the reaction of adipic acid and myristyl alcohol containing from one to four repeating propoxy groups and from one to twenty repeating ethoxy groups wherein the compound is di-PPG-2 myreth-10 adipate having the formula:

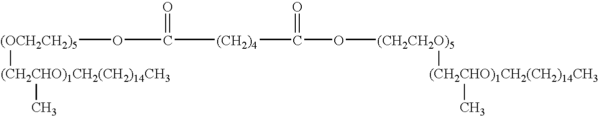

4. The Triester.

The compound may also be a triester formed by (i) the reaction of aromatic tricarboxylic acid and an aliphatic or aromatic alcohol, or (ii) a polyol having primary hydroxyl groups, with an aliphatic carboxylic acid. The triester may have one or more alkyl ether groups as defined above.

One preferred ester is (i), above, where the tricarboxylic acid comprises trimellitic acid, and the aliphatic alcohol has from one to forty, preferably one to twenty eight carbon atoms. More preferred is where the aliphatic alcohol is selected from the group consisting of butyl alcohol, tridecyl alcohol, and mixtures thereof. Most preferred is where the triester is formed by the reaction of trimellitic acid and straight or branched butyl alcohol, one of said preferred Compounds being tributyl trimellitate, and having the formula:

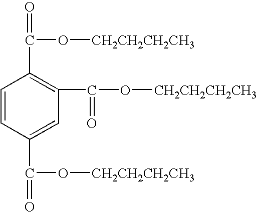

wherein one or more of the butyl groups as depicted above may be straight or branched chain.

The triester may also be formed by the reaction of a polyol having primary hydroxyl groups with an aliphatic carboxylic acid where either of the alcohol or acid comprises from about 1 to 100 carbon atoms. The term “polyol” means an alcohol having two or more hydroxyl groups, the number of carbon atoms ranging from about 3 to 30, preferably 3 to 10, most preferably about 6. The term “primary hydroxyl groups” means that the hydroxyl groups are bonded to the end of an alkyl chain in the alcohol molecule. In contrast, a secondary alcohol is one where the hydroxyl group is attached to the intermediate carbon atom in the alcohol and the alcohol has two alkyl chains; and a tertiary alcohol one where the hydroxyl group is attached to an intermediate carbon atom in the alcohol and the alkyl chain is branched, having three alkyl chains.

Most preferred is where the C₃₋₁₀ aliphatic alcohol is the primary alcohol trimethylolpropane and the C₁₋₁₀ carboxylic acid is propionic acid, and the triester is trimethylolpropane tripropionate.

The nail enamel composition may contain only one or any mixture of the Compounds. The Compounds may reduce the hardness of the film forming polymers present in the nail enamel composition, which in turn has a positive impact on nail enamel wear.

D. Other Ingredients

The nail enamel compositions of the invention may contain a variety other ingredients that will enhance the beneficial properties, such as UV absorbers, plasticizers, defoaming agents, pigments, powders, suspending agents, and so on.

1. Pigments

The nail enamel compositions of the invention may be pigmented or clear. If pigmented, generally about 0.1-30% by weight of the total composition, preferably about 0.5-20%, more preferably about 1-15% of pigment is suggested. Pigments suitable for use in nail enamel compositions are well known and include iron oxides, D&C and FD&C colors, titanium dioxide, and the like. The pigments may be treated or coated with agents that modify the surface properties, such as silicones. Examples of silicone treated pigments that can be used in the compositions of the invention are set forth in U.S. Pat. No. 4,832,944, which is hereby incorporated by reference in its entirety.

2. Suspending Agents

If the nail enamel compositions of the invention contain pigments, it is desirable to also incorporate from about 0.1-15% by weight of the total composition of a suspending agent which acts to suspend the pigments in the formulation. Suitable suspending agents are montmorillonite minerals or synthetic derivatives thereof. These montmorillonite minerals or synthetic metal silicate gelling agents may be reacted with other compounds such as unsubstituted quarternary ammonium compounds or quaternary ammonium compounds substituted with one or more fatty chains. Examples of such materials include bentonite, hectorite, attapulgite, bentones, and the like. Examples of quaternized derivatives thereof include stearalkonium bentonite, etc.

Also suitable are polymeric compounds known as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl.

3. Defoaming Agents

It may also be desireable to include from about 0.1-20% by weight of the total composition, of one or more defoaming agents in the composition. The defoaming agent reduces the tendency of the nail enamel to bubble when applied, and also improves the surface finish. Preferrred are silicones, either polydimethylsiloxanes or silicone glycol copolymers (also referred to as dimethicone copolyols). Silicone glycol copolymers, which may be used in the compositions of the invention are pblymethylsiloxanes wherein a portion of the methylsiloxane units are substituted with polyalkylene glycol ether moieities. Preferred is wherein about 60-90% of the polymer (the percentage being based on the number of monomer units), of the compound is polydimethylsiloxane or polyhydrogen methylsiloxane and 30-40% of the compound (the percentage being based upon the number of monomer units) is di-methyl or hydrogen-methyl siloxane units substituted with polyalkylene glycol ethers. Most preferred are silicone glycol copolymers having a viscosity ranging from 1 to 500,000, preferably 1 to 2,000 centipoise at 25° C., a specific gravity ranging from 0.80 to 1.030 at 25° C., and comprise approximately 80% dimethylsiloxane units and 20% propylene oxide substituted methyl siloxane units. Silicone glycol copolymers having this description are commercially available from a variety of sources including Dow Corning under the tradenames Dow Corning Additive 3, 7, 11, 14, 18, 21, 24, 26, 28, 29, 51, 54, 56, 57, and 1248.

4. UV Absorbers or Sunscreens

In many cases it is desirable to incorporate UV absorbers into the nail enamel composition. The UV absorbers will block UV radiation within a certain range and protect the nail from UV light. The UV absorbers may be UVA or UVB sunscreen.

(a) UVA Sunscreens

The term “UVA sunscreen” means a chemical compound that blocks UV radiation in the wavelength range of about 320 to 400 nm. Preferred UVA sunscreens are dibenzoylmethane compounds having the general formula:

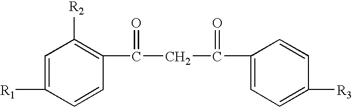

wherein R₁ is H, OR and NRR wherein each R is independently H, C₁₋₂₀ straight or branched chain alkyl; R₂ is H or OH; and R₃ is H, C₁₋₂₀ straight or branched chain alkyl.

Preferred is where R₁ is OR where R is a C₁₋₂₀ straight or branched alkyl, preferably methyl; R₂ is H; and R₃ is a C₁₋₂₀ straight or branched chain alkyl, more preferably, butyl.

Examples of suitable UVA sunscreen compounds of this general formula include 4-methyldibenzoylmethane, 2-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′diisopropylbenzoylmethane, 4-tert-butyl-4′-methoxydibenzoylmethane, 4,4′-diisopropylbenzoylmethane, 2-methyl-5-isopropyl-4′-methoxydibenzoylmethane, 2-methyl-5-tert-butyl-4′-methoxydibenzoylmethane, and so on. Particularly preferred is 4-tert-butyl-4′-methoxydibenzoylmethane, also referred to as Avobenzone. Avobenzone is commercial available from Givaudan-Roure under the trademark Parsol 1789, and Merck & Co. under the tradename Eusolex 9020.

The nail enamel compositions of the invention may contain from about 0.001-20%, preferably 0.005-5%, more preferably about 0.005-3% by weight of the composition of UVA sunscreen.

(b). UVB Sunscreens

The term “UVB sunscreen” means a compound that blocks UV radiation in the wavelength range of from about 290 to 320 nm. A variety of UVB chemical sunscreens exist including α-cyano-β,β′-diphenyl acrylic acid esters as set forth in U.S. Pat. No. 3,215,724, which is hereby incorporated by reference in its entirety. One particular example of a α-cyano-β,β′-diphenyl acrylic acid ester is Octocrylene, which is 2-ethylhexyl 2-cyano-3,3-diphenylacrylate. In certain cases the composition may contain no more than about 10% by weight of the total composition of octocrylene. Suitable amounts range from about 0.001-10% by weight. Octocrylene may be purchased from BASF under the tradename Uvinul N-539.

Other suitable sunscreens include benzylidene camphor derivatives as set forth in U.S. Pat. No. 3,781,417, which is hereby incorporated by reference in its entirety. Such benzylidene camphor derivatives have the general formula:

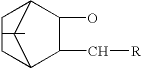

wherein R is p-tolyl or styryl, preferably styryl. Particularly: preferred is 4-methylbenzylidene camphor, which is a lipid soluble UVB sunscreen compound sold under the tradename Eusolex 6300 by Merck.

Also suitable are cinnamate derivatives having the general formula:

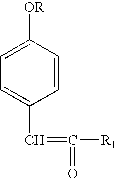

wherein R and R₁ are each independently a C₁₋₂₀ straight or branched chain alkyl. Preferred is where R is methyl and R₁ is a branched chain C₁₋₁₀, preferably C₈ alkyl. The preferred compound is ethylhexyl methoxycinnamate, also referred to as Octoxinate or octyl methoxycinnamate. The compound may be purchased from Givaudan Corporation under the tradename Parsol MCX, or BASF under the tradename Uvinul MC 80. Also suitable are mono-, di-, and triethanolamine derivatives of such methoxy cinnamates including diethanolamine methoxycinnamate. Cinoxate, the aromatic ether derivative of the above compound is also acceptable. If present, the Cinoxate should be found at no more than about 3% by weight of the total composition.

Also suitable as the UVB screening agents are various benzophenone derivatives having the general formula:

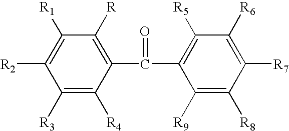

R through R₉ are each independently H, OH, NaO₃S, SO₃H, SO₃Na, Cl, R″, OR″ where R″ is C₁₋₂₀ straight or branched chain alkyl. Examples of such compounds include Benzophenone 1, 2, 3, 4, 5, 6, 7, 8, 9, 10, 11, and 12. Particularly preferred is where the benzophenone derivative is Benzophenone 3 (also referred to as Oxybenzone) and Benzophenone 4 (also referred to as Sulisobenzone), Benzophenone 5 (Sulisobenzone Sodium), and the like. Most preferred is Benzophenone 3.

Also suitable are certain menthyl salicylate derivatives having the general formula:

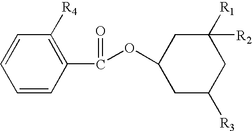

wherein R₁, R₂, R₃, and R₄ are each independently H, OH, —NH₂, or C₁₋₂₀ straight or branched chain alkyl. Particularly preferred is where R₁, R₂, and R₃ are methyl and R₄ is hydroxyl or NH₂, the compound having the name homomenthyl salicylate (also known as Homosalate) or menthyl anthranilate. Homosalate is available commercially from Merck under the tradename Eusolex HMS and menthyl anthranilate is commercially available from Haarmann & Reimer under the tradename Heliopan. If present, the Homosalate should be found at no more than about 15% by weight of the total composition.

Various amino benzoic acid derivatives are suitable UVB absorbers including those having the general formula:

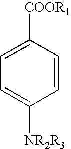

Wherein R₁, R₂, and R₃ are each independently H, C₁₋₂₀ straight or branched chain alkyl which may be substituted with one or more hydroxy groups. Particularly preferred is wherein R₁ is H or C₁₋₈ straight or branched alkyl, and R₂ and R₃ are H, or C₁₋₈ straight or branched chain alkyl. Particularly preferred are PABA, ethyl hexyl dimethyl PABA (Padimate O), ethyldihydroxypropyl PABA, and the like. If present Padimate O should be found at no more than about 8% by weight of the total composition.

Salicylate derivatives are also acceptable UVB absorbers. Such compounds have the general formula:

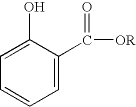

wherein R is a straight or branched chain alkyl, including derivatives of the above compound formed from mono-, di-, or triethanolamines. Particular preferred are octyl salicylate, TEA-salicylcate, DEA-salicylate, and mixtures thereof.

Generally, the amount of the UVB chemical sunscreen present may range from about 0.001-45%, preferably 0.005-40%, more preferably about 0.01-35% by weight of the total composition.

5. Preservatives

The composition of the invention may contain one or more preservatives. Suitable ranges include about 0.001-8%, preferably about 0.01-6%, more preferably about 0.05-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and the like.

II. The Method

The invention also comprises a method for reducing the hardness of one or more film forming polymers in a nail enamel composition, and/or improving nail enamel wear comprising adding to the nail enamel composition a Compound selected from the group consisting of:

(a) a monoester containing at least one alkyl ether group,

(b) a monoester of benzoic acid and a long chain alcohol or ester alkanol, or fatty oil,

(c) a diester formed by the reaction of (i) a dicarboxylic acid where the two carboxylic acid groups are separated by a straight or branched chain alkyl group or an aryl group, and (ii) an aliphatic or aromatic alcohol; said diester further comprising at least one alkyl ether group.

(d) a triester formed by (i) the reaction of aromatic tricarboxylic acid and an aliphatic or aromatic alcohol, or (ii) the reaction of a polyol having primary hydroxyl groups and an aliphatic carboxylic acid; and

(e) and mixtures thereof.

Addition of the Compound, in the ranges set forth herein, will cause the film formed by the film forming polymer to exhibit a reduced hardness. It will also improve the wear of the nail enamel composition. The term “wear” means the amount of time the nail enamel can be worn on the nails without requiring touch ups or a new application. Generally wear is measured by tests such as the tip wear index test which involves painting the nails with the nail enamel and having trained evaluators assess the degree of nail enamel wear on a daily basis for the desired period of time.

Generally, hardness is measured by analysis of Sward hardness according to ASTM test methods, in particular ASTM test method D2134-93(2001). In particular, one method for performing the Sward Hardness analysis is set forth as follows.

### Sward Hardness Test

Reagents and Equipment

Acetone

Sward Hardness Rocker

Glass plates

Leveling table

Test nail enamel

6 ml. bird applicator

Calibration of Sward Hardness Rocker

The Sward Hardness Rocker (SHR) is calibrated. Plate glass is placed on a leveling table and the SHR is placed on the glass and leveled. Oscillation of the SHR is started so that the bubble in the left hand tube slightly overlaps the mark. The count is started, beginning with zero, when the bubble in the left hand tube just fails to reach the mark. The count is stopped when the bubble in the right hand tube just fails to reach the mark. The SHR should make 50 complete oscillations in 60+/−0.5 seconds. If the number of swings is not 50, the left hand bubble tube is adjusted to give the correct value. The time for 50 swings can be changed by adjustments in the position of the weight on the vertical screw. The properly calibrated SHR shows a decrease in amplitude of 6 degrees from the vertical, after 50 swings on the glass, taken between 22 and 16 degrees from the vertical.

If desired, an automatic, or digital, SHR may also be used. Suitable digital SHR's are made by Gardner Co., Inc., Pompano Beach Fla., under the brand name Gardco, Gardner/Sward Hardness Rocker Model GSI. In this case the automatic, or digital SHR is set for 50 rocks, and it records the number of rocks and displays the reading. This reading is multiplied by two to arrive at the final result.

Sample Test

For each test sample a clear glass plate and a 6 mil bird applicator are cleaned with acetone. A suitable size puddle of the test composition is poured side by side on the glass plate and drawn down immediately to a uniform film with no ripples, uneven spots, specks or lumps present. The samples were allowed to dry for 2, 24, and 48 hours. The Sward Hardness test is conducted after the samples were dried for the time periods mentioned.

After the SHR is calibrated, it is placed on the plate in a way that does not mar the film. The SHR is tilted to the left so that both bubbles are to the right, then gently released so that it oscillates on the film. The right bubble is observed. When it just stops going past the mark, the number of times the left bubble oscillates from right to left is counted. One oscillation is counted for each time the left hand bubble touches the left wall of the tube. The left bubble's first partial oscillation past the mark is estimated to the nearest 0.25 oscillation. No additional partial oscillations are counted after the first partial oscillation.

The Sward Hardness of the film is calculated using the following equation:

N×2=R

wherein N=number of oscillations

R=Sward Hardness

2=factor

Typically, in nail enamels that do not contain any of the Compound, the Sward Hardness value of the film will range from about 45-50 after two hours. This provides a very brittle film that chips readily and is not appropriate for commercial use.

However, in the compositions of the invention, the Sward Hardness values will be considerably reduced to more appropriate levels. Preferred ranges for Sward Hardness in the enamel of the invention are set forth below:

| Sward Hardness Value | | | Measurement Time |
|---|---|---|---|
| Preferred Range | More Preferred Range | Most Preferred Range | (hours after film dry) |
| 2-14 | 2-10 | 2-8 | 2 |
| 2-18 | 2-16 | 2-14 | 2-5 |
| 2-26 | 2-20 | 4-16 | 2-12 |
| 6-30 | 6-24 | 6-18 | 24 |

In the most preferred method of the invention, the Sward Hardness of the dried nail enamel film reaches the optimal Sward Hardness value ranging from about 2-14 within 2 hours after the nail enamel film has been applied to the nails.

The nail enamel composition of the invention is applied to the nails in the usual manner. While a base coat is not necessary, when it is used the wear may be improved. In this case, a first base coat is applied to the nails and allowed to dry. This is followed by one or two color coats. Ideally a clear topcoat should be applied on top of the color coat to provide optimum protection to the nail enamel finish.

The addition of the Compound will cause the nail enamel film to exhibit reduced hardness. This, in turn, will improve nail enamel wear.

The invention will be further described in connection with the following examples which are set forth for the purposes of illustration only.

### EXAMPLE 1

Compounds were tested for their ability to modify the hardness of a nail enamel film. Nail enamel compositions were prepared as follows:

| | w/w % |
|---|---|
| Cellulose acetate propionate | 15.00 |
| N-propyl acetate | 21.00 |
| N-butyl acetate | 24.00 |
| Ethyl acetate | 28.00 |
| Compound* | 12.00 |

*1 - N-butyl acetate (control without Compound)
2 - tributyl trimellitate
3 - di-PPG-3 myristyl ether adipate
4 - di-PPG-2 myreth-10 adipate
5 - propylene glycol myristyl ether acetate
6 - dipropylene glycol isoceteth-20
7 - propylene glycol isoceteth-3 acetate
8 - trimethylolpropane tripropionate

The compositions were applied to glass plates and allowed to dry, after which the Sward Hardness and Cross Hatch tests were conducted.

Sward Hardness: for each test sample a clear glass plate and a 6 mil bird applicator were cleaned with acetone. A suitable size puddle of the composition was poured next to the standard (#1) side by side on the glass plate and drawn down immediately to a uniform film with no ripples, uneven spots, specks or lumps present. The Sward Hardness test was conducted after the samples were allowed to dry for 2, 24, and 48 hours usin a digital SHR made by Gardner Co., Inc, Pompano Beach, Fla., under the brand name Gardco, Garner/Sward Hardness Rocker Model GSI.

The Sward Hardness of the film was calculated using the following equation:

N×2=R

wherein N=number of oscillations

R=Sward Hardness

2=factor

Cross Hatch:

The cross hatch test was conducted using ASTM Test Method D 3359-87, which is hereby incorporated by reference.

The results were as follows:

| | | | | | | | | | | | Cross | |
|---|---|---|---|---|---|---|---|---|---|---|---|---|
| | Sward Hardness | | | | | | | | | | Hatch | |
| | Hours | | | Days | | | | | | | (days) | |
| *Compound | 2 | 24 | 48 | 3 | 5 | 7 | 8 | 10 | 12 | 14 | 8 | 14 |
| 1 | 46 | 46 | 50 | — | 47 | 46 | 49 | — | 41 | 43 | 1B | 1B |
| 2 | 12 | 16 | 12 | 14 | 12 | 14 | 14 | 14 | 14 | 14 | 0B | 0B |
| 3 | 22 | 23 | 22 | 22 | 22 | 22 | 22 | 22 | 23 | 26 | 1B | 2B |
| 4 | 8 | 11 | 9 | 9 | 10 | 8 | 8 | 6 | — | 7 | 0B | 0B |
| 5 | 21 | 24 | 25 | 25 | 25 | 25 | 23 | 24 | 22 | 22 | 4B | 4B |
| 6** | 8 | 8 | — | 0 | 0 | | | | | | 0B | |
| 7 | 18 | 20 | 22 | 20 | 21 | 21 | 19 | 19 | 18 | 17 | 4B | 2B |
| 8 | 2 | 4 | 4 | — | 2 | 4 | 4 | — | 4 | 4 | 1B | 1B |

**test discontinued due to crystallization

### EXAMPLE 2

Another set of Compounds were tested for their ability to modify the hardness of a nail enamel film. Nail enamel compositions were prepared as follows:

| | w/w % |
|---|---|
| ¼ sec. nitrocellulose | 23.0 |
| n-butyl acetate | 20.0 |
| Ethyl acetate | 41.0 |
| Compound* | 16.0 |

*1 PPG-3 benzyl myristate
2 Isostearyl benzoate
3 Ethylhexyl benzoate
4 Mixture of C12-15 alkyl benzoate, dipropylene glycol dibenzoate, PPG-15 stearyl ether benzoate
5 Cetyl ricinoleate benzoate
6 Castor oil benzoate.

| | | | | | | | | Cross | |
|---|---|---|---|---|---|---|---|---|---|
| | Sward Hardness | | | | | | | Hatch | |
| | Hours | | | Days | | | | (days) | |
| *compound | 2 | 24 | 48 | 5 | 7 | 12 | 14 | 5 | 14 |
| 1 | 12-14 | 12-14 | 12-14 | 12-14 | 18 | 16-18 | 16-18 | 5B | 2B |
| 2 | 16 | 22 | 22 | 22 | 24 | 22-24 | 22 | 5B | 5B |
| 3 | 10-14 | 14-16 | 16 | 18 | 18-20 | 18-24 | 22 | 5B | 4B |
| 4 | 16 | 18 | 20 | 20-24 | 22 | 22 | 22 | 5B | 5B |
| 5 | 14-16 | 18-20 | 20 | 18-22 | 20 | 18-22 | 22 | 4B | 5B |
| 6 | 14-16 | 18-22 | 16-20 | 16-20 | 18 | 18-20 | 18-20 | 5B | 4B |

The above results illustrate that nail enamels containing the Compounds were tested, they exhibited Sward hardness values that were in the preferred ranges. The cross-hatch values indicate that the films are flexible and exhibit adhesion to glass, which is one indication of how the product performs on the nails.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A nail enamel composition comprising at least one solvent, at least one film forming polymer, and at least one compound selected from the group consisting of (“Compound”):
(a) a monoester containing at least one alkyl ether group,
(b) a monoester of benzoic acid and a long chain alcohol or ester alkanol, or fatty oil,
(c) a diester formed by the reaction of a dicarboxylic acid where the two carboxylic acid groups are separated by a straight or branched chain alkyl group or an aryl group; and an aliphatic or aromatic alcohol; said diester further comprising at least one alkyl ether group,
(d) a triester formed by (i) the reaction of aromatic tricarboxylic acid and an aliphatic or aromatic alcohol, or (ii) the reaction of a polyol having primary hydroxyl groups and an aliphatic carboxylic acid; and
(e) and mixtures thereof.

2. The composition of claim 1 wherein the Compound is a monoester containing at least one alkyl ether group.

3. The composition of claim 2 wherein the alkyl ether group is a repeating alkyl ether group.

4. The composition of claim 2 wherein the monoester containing at least one alkyl ether group is formed by the reaction of a C₁₋₁₀₀ aliphatic or aromatic carboxylic acid and an aliphatic or aromatic alcohol containing the at least one alkyl ether group.

5. The composition of claim 4 wherein the at least one alkyl ether group is ethoxy, propoxy, isopropoxy, butoxy, isobutoxy, or combinations thereof.

6. The composition of claim 5 wherein the monoester is formed by the reaction of a C₁₋₄₈ aliphatic or aromatic carboxylic acid and a C₁₋₁₀₀ aliphatic alcohol having at least one ethoxy, propoxy, isopropoxy, butoxy, or isobutoxy group, or combinations thereof.

7. The compound of claim 6 wherein the aliphatic alcohol has one to four repeating propoxy groups and one to twenty repeating ethoxy groups.

8. The composition of claim 7 wherein the monoester is the reaction product of a C₁₋₂₀ aliphatic carboxylic acid and a C₁₋₁₀₀ aliphatic alcohol having one to four repeating propoxy groups and one to twenty repeating ethoxy groups.

9. The composition of claim 8 wherein the C₁₋₂₀ aliphatic carboxylic acid is acetic acid and the aliphatic alcohol is cetyl alcohol having one to four repeating propoxy groups and one to twenty repeating ethoxy groups.

10. The composition of claim 9 wherein the monoester is formed by the reaction of acetic acid and cetyl alcohol having one propoxy group and three ethoxy groups, and said compound is propylene glycol isoceteth-3 acetate.

11. The composition of claim 6 wherein the monoester is formed by the reaction of acetic acid and myristyl alcohol having at least one ethoxy, propoxy, isopropoxy, butoxy, or isobutoxy group.

12. The composition of claim 11 wherein the monoester is formed by the reaction of acetic acid and myristyl alcohol having at least one ethoxy group and said Compound is propylene glycol myristyl ether acetate.

13. The composition of claim 2 wherein the monoester is formed by the reaction of an aromatic acid and a C₁₋₁₀₀ aliphatic alcohol containing at least one alkyl ether group.

14. The composition of claim 13 wherein the aromatic acid is benzoic acid and the alcohol containing at least one alkyl ether group is a C₁₋₃₀ alcohol having one or more repeating alkyl ether groups.

15. The composition of claim 14 wherein the repeating alkyl ether groups are ethoxy, propoxy, isopropoxy, butoxy, or isobutoxy, or mixtures thereof.

16. The composition of claim 15 wherein the monoester is the reaction product of benzoic acid and a C₆₋₅₀ fatty alcohol having from 1 to 20 repeating propoxy groups.

17. The composition of claim 16 wherein the compound is PPG-15 stearyl ether benzoate.

18. The composition of claim 2 wherein the monoester is formed by the reaction of benzyl alcohol and myristic acid where either the alcohol or the acid has substituted from 1 to 20 repeating propoxy groups.

19. The composition of claim 18 wherein the Compound is PPG-3 benzyl myristate.

20. The composition of claim 1 wherein the compound is a monoester of benzoic acid and a long chain alcohol or ester alkanol, or fatty oil.

21. The composition of claim 20 wherein the monoester is the monoester of benzoic acid and a long chain alcohol.

22. The composition of claim 21 wherein the long chain alcohol has from about 6 to 50 carbon atoms.

23. The composition of claim 22 wherein the alcohols are stearyl alcohol, ethylhexyl alcohol, C12-15 alcohols, myristyl alcohol, cetyl alcohol, isostearyl alcohol, oleyl alcohol, or mixtures thereof.

24. The composition of claim 23 wherein the monoester is the ester of benzoic acid and isostearyl alcohol and the compound is isostearyl benzoate.

25. The composition of claim 23 wherein the monoester is the ester of benzoic acid and ethylhexyl alcohol and the compound is ethylhexyl benzoate.

26. The composition of claim 23 wherein the monoester is an ester of benzoic acid and C12-15 alcohols and the compound is C12-15 alkyl benzoate.

27. The composition of claim 20 wherein the monoester is the ester of benzoic acid and an ester alkanol.

28. The composition of claim 27 wherein the ester alkanol is a long chain ester alkanol.

29. The composition of claim 29 wherein the ester alkanol has a mixture of saturated and unsaturated carbon atoms.

30. The composition of claim 28 wherein the ester alkanol is formed by the reaction of a fatty C₆₋₅₀ carboxylic acid and a fatty C₆₋₅₀ alcohol where the carboxylic acid or the alcohol contains at least one or more hydroxyl groups.

31. The composition of claim 30 wherein the benzoic acid ester is the reaction product of benzoic acid and cetyl ricinoleic alcohol and the compound is cetyl ricinoleate benzoate.

32. The composition of claim 30 wherein the benzoic acid ester is the reaction product of benzoic acid and cetylstearyl alcohol or cetyl isostearyl alcohol.

33. The composition of claim 20 wherein the monoester is an ester of benzoic acid and a fatty oil.

34. The composition of claim 33 wherein the fatty oil is castor oil.

35. The composition of claim 34 wherein the monoester is castor oil benzoate.

36. The composition of claim 1 wherein the Compound is a diester formed by the reaction of a dicarboxylic acid where the two carboxylic acid groups are separated by a straight or branched chain alkyl group or an aryl group; and an aliphatic or aromatic alcohol and said diester further comprising at least one alkyl ether group.

37. The composition of claim 36 wherein the diester is formed by the reaction of a dicarboxylic acid where the two carboxylic acid groups are separated by a straight or branched chain alkyl group.

38. The composition of claim 37 wherein the aliphatic alcohol contains at least one alkyl ether group.

39. The composition of claim 38 wherein the two carboxylic acid groups are separated by a straight or branched chain alkyl group containing from one to forty carbon atoms.

40. The composition of claim 49 wherein the aliphatic alcohol contains repeating alkyl ether groups which are ethoxy, propoxy, isopropoxy, butoxy, isobutoxy, or mixtures thereof.

41. The composition of claim 40 wherein the diester is the reaction product of a dicarboxylic acid where the two carboxylic acid groups are separated by a C₁₋₁₀ straight chain alkyl and the alkyl ether group on the aliphatic alcohol is one or more propoxy groups.

42. The composition of claim 41 wherein both carboxylic acid groups of the dicarboxylic acid are reacted with an aliphatic alcohol containing repeating alkyl ether groups which are ethoxy, propoxy, isopropoxy, or mixtures thereof.

43. The composition of claim 42 wherein the carboxylic acid is adipic acid and the aliphatic alcohol is myristyl alcohol containing from one to four repeating propoxy groups and wherein the compound is di-PPG-3 myristyl ether adipate.

44. The composition of claim 43 wherein the diester is formed by the reaction of adipic acid and myristyl alcohol containing from one to four repeating propoxy groups and from one to twenty ethoxy groups wherein the Compound is di-PPG-2 myreth-10 adipate.

45. The composition of claim 1 where the Compound is a triester formed by (i) the reaction of an aromatic tricarboxylic acid and an aliphatic or aromatic alcohol, or (ii) the reaction of a polyol having primary hydroxyl groups and an aliphatic carboxylic acid.

46. The composition of claim 45 wherein the Compound is formed by the reaction of an aromatic tricarboxylic acid and an aliphatic or aromatic alcohol.

47. The composition of claim 46 wherein the tricarboxylic acid is trimellitic acid.

48. The composition of claim 47 wherein the aliphatic alcohol has from one to forty carbon atoms.

49. The composition of claim 48 wherein the aliphatic alcohol has from one to twenty eight carbon atoms.

50. The composition of claim 49 wherein the aliphatic alcohol is butyl alcohol, tridecyl alcohol, and mixtures thereof.

51. The composition of claim 49 wherein the triester is formed by the reaction of trimellitic acid and butyl alcohol, and said compound is tributyl trimellitate.

52. The composition of claim 45 wherein the triester is formed by the reaction of a tertiary aliphatic or aromatic alcohol and an aliphatic or aromatic carboxylic acid.

53. The composition of claim 52 wherein the alcohol is a tertiary aliphatic alcohol having 1-10 carbon atoms.

54. The composition of claim 53 wherein the carboxylic acid is an aliphatic carboxylic acid having from 1-10 carbon atoms.

55. The composition of claim 54 wherein the triester is the reaction product of trimethylolpropane and propionic acid and the Compound is trimethylolpropionate.

56. The composition of claim 1 further comprising from about 0.1-30% by weight of the total composition of pigment.

57. The composition of claim 1 further comprising about 0.1-30% by weight of the total composition of suspending agent.

58. The composition of claim 1 further comprising 0.1-20% by weight of the total composition of one or more defoaming agents.

59. The composition of claim 1 further comprising 0.001-45% by weight of the total composition of UV absorbers.

60. A method for reducing the hardness of one or more film forming polymers in a nail enamel composition, and/or improving nail enamel wear comprising adding to the nail enamel composition a compound selected from the group consisting of:
(a) a monoester containing at least one alkyl ether group,
(b) a monoester of benzoic acid and a long chain alcohol or ester alkanol, or fatty oil,
(c) a diester formed by the reaction of a dicarboxylic acid where the two carboxylic acid groups are separated by a straight or branched chain alkyl group or an aryl group, and an aliphatic or aromatic alcohol and said diester further comprising at least one alkyl ether group.
(d) a triester formed by (i) the reaction of aromatic tricarboxylic acid and an aliphatic or aromatic alcohol, or (ii) the reaction of a polyol having primary hydroxyl groups with an aliphatic carboxylic acid; and
(e) and mixtures thereof.
